(19) United States
(12) Patent Application Publication
(10) Pub. No.: US 2023/0105440 A1
(43) Pub. Date: Apr. 6, 2023

(54) ANTI-THROMBOGENIC COATING

(71) Applicant: Medtronic, Inc., Minneapolis, MN (US)

(72) Inventors: Valentino J. Tramontano, Brockton, MA (US); Sean A. McDermott, Weymouth, MA (US)

(21) Appl. No.: 17/495,513

(22) Filed: Oct. 6, 2021

(51) Int. Cl. A61L 33/06; A61M 25/00; A61L 29/08; A61L 29/14; A61L 29/16; A61K 38/36

(52) U.S. Cl. CPC A61L 33/062; A61K 38/363; A61L 29/16; A61L 29/085; A61L 29/148; A61M 25/003; A61M 25/0009; A61M 25/0045; A61L 2300/42; A61L 2300/252; A61L 2300/602; A61L 2300/622; A61M 2025/0031; A61M 2205/0238

(57) ABSTRACT

An example medical device includes a vascular device, such as a catheter, and an anti-thrombogenic coating on a surface of the vascular device, such as a surface likely to contact blood. The anti-thrombogenic coating includes one or more peptides configured to interact with fibrinogen in the blood, such as a first type of peptides configured to bind to fibrinogen a second type of peptides configured to inhibit conversion of fibrinogen to fibrin. The anti-thrombogenic coating also includes a polymer, such as a hydrocolloid polymer, a tunable polyethylene glycol (PEG), or other controlled release polymer configured to control release of the one or more peptides and maintain a concentration of the peptides at the surface of the anti-thrombogenic coating above a minimum inhibitory concentration, thereby inhibiting thrombin formation on the intravascular medical device.

### TECHNICAL FIELD

This disclosure relates to anti-thrombogenic coatings, e.g., for hemodialysis catheters or other medical devices.

### BACKGROUND

A medical catheter may be used to withdraw and introduce fluids to and from body cavities, ducts, blood vessels, and other hollow anatomical structures of a patient. As an example, a catheter may be used in hemodialysis procedures, in which blood is withdrawn from a blood vessel of a patient for treatment and subsequently returned to the blood vessel for circulation.

### SUMMARY

The present disclosure describes vascular devices that include an anti-thrombogenic coating that inhibits fibrin and/or thrombus formation on the vascular device. The anti-thrombogenic coating may be applied to one or more surfaces of a vascular device that may contact blood. Blood includes fibrinogen, which may form a thrombus on exposed surfaces of a vascular device both by depositing on the surfaces and binding to platelets and by converting to soluble fibrin and polymerizing with the bound platelets. The anti-thrombogenic coating may release one or more peptides that interact with fibrinogen in the blood at the surface of the vascular device to inhibit thrombus formation, such as by binding to the fibrinogen and/or inhibiting polymerization of fibrinogen-derived monomers, such as fibrin monomers. In some examples, the anti-thrombogenic coating maintains the concentration of the peptides at the surface of the anti-thrombogenic coating above a minimum inhibitory concentration. As a result, the anti-thrombogenic coating may inhibit thrombus formation in portions of the vascular device otherwise prone to thrombus formation.

The anti-thrombogenic coating may control the availability of the peptides at the surface of the coating using a variety of controlled release and/or exposure mechanisms. In one example, the anti-thrombogenic coating includes a bioabsorbable polymer matrix that releases the peptide at a particular release rate corresponding to dissolution of the bioabsorbable polymer matrix. The polymer matrix may exhibit tunable physicochemical properties such as permeability, molecular diffusivity, and degradation rate, to release the peptide at a particular release rate. In another example, the anti-thrombogenic coating includes peptides encapsulated in bioabsorbable polymer shells as microspheres and dispersed in a polymer matrix, such as a hydrocolloid polymer matrix. In these various ways, the anti-thrombogenic coating may maintain a sufficient concentration of the peptides at the surface of the anti-thrombogenic coating for a long period of time to inhibit thrombus formation.

The anti-thrombogenic coatings may be applied to a variety of vascular devices used in a variety of systems. In some examples, one or more surfaces of a catheter, such as a hemodialysis catheter, includes the anti-thrombogenic coating. For example, at least a distal portion of a hemodialysis catheter may include the anti-thrombogenic coating to reduce thrombin formation during long periods in which the hemodialysis catheter is implanted into a patient or otherwise positioned in the vasculature of the patient.

In some examples, a medical device includes a vascular device and an anti-thrombogenic coating on a surface of the vascular device. The anti-thrombogenic coating includes one or more peptides and a polymer matrix. The one or more peptides are configured to interact with fibrinogen and/or fibrinogen-derived proteins in blood. The polymer matrix is configured to control availability of the one or more peptides at a surface of the anti-thrombogenic coating.

In some examples, a hemodialysis system includes a hemodialysis catheter assembly configured to fluidically couple to a hemodialysis machine. The hemodialysis catheter assembly includes a catheter and an anti-thrombogenic coating on a surface of the catheter. The catheter includes an elongated body and defines an aspiration lumen and a perfusion lumen. The anti-thrombogenic coating includes one or more peptides and a polymer matrix. The one or more peptides are configured to interact with fibrinogen or fibrinogen-derived proteins in blood. The polymer matrix is configured to control availability of the one or more peptides at a surface of the anti-thrombogenic coating.

In some examples, a method includes forming an anti-thrombogenic coating on a surface of a vascular device. The anti-thrombogenic coating includes one or more peptides and a polymer matrix. The one or more peptides are configured to interact with fibrinogen and/or fibrinogen-derived proteins in blood. The polymer matrix is configured to control availability of the one or more peptides at a surface of the anti-thrombogenic coating.

The details of one or more examples are set forth in the accompanying drawings and the description below. Other features, objects, and advantages will be apparent from the description and drawings, and from the claims.

### BRIEF DESCRIPTION OF DRAWINGS

FIG. 1A is a conceptual diagram of an example hemodialysis system that includes a hemodialysis catheter and a patient.

FIG. 1B is a cross-sectional conceptual diagram of the example hemodialysis catheter of FIG. 1A that includes an anti-thrombogenic coating.

FIG. 1C is an expanded view of a distal portion of the catheter assembly of FIG. 1B that includes an anti-thrombogenic coating.

FIG. 2A is a conceptual diagram illustrating fibrinogen-derived thrombus formation.

FIG. 2B is a conceptual diagram illustrating inhibition of fibrinogen-derived thrombus formation using an anti-thrombogenic coating.

FIG. 3A is a conceptual diagram illustrating an anti-thrombogenic coating that includes a diffusion-controlled polymer matrix.

FIG. 3B is a conceptual diagram illustrating an anti-thrombogenic coating that includes a biodegradable polymer matrix.

FIG. 3C is a conceptual diagram illustrating an anti-thrombogenic coating that includes microspheres in a biodegradable polymer matrix.

FIG. 4 is a flowchart illustrating an example technique for forming a medical device that includes a vascular device and an anti-thrombogenic coating.

Like reference characters denote like elements throughout the description and figures.

### DETAILED DESCRIPTION

The present disclosure describes example anti-thrombogenic coatings that inhibit fibrin and thrombus formation on a vascular device, and vascular devices that include such coatings. In some examples, the anti-thrombogenic coating is applied to one or more surfaces of a vascular device that may contact blood. Blood includes fibrinogen, which forms a thrombus on exposed surfaces both by depositing on surfaces and binding to platelets and by converting to soluble fibrin and polymerizing with the bound platelets. The anti-thrombogenic coating may release one or more peptides that interact with fibrinogen in the blood at the surface of the vascular device to inhibit thrombus formation, such as by binding to the fibrinogen and/or inhibiting polymerization of fibrinogen-derived monomers, such as fibrin monomers. The anti-thrombogenic coating may maintain the concentration of the peptides at the surface of the anti-thrombogenic coating above a minimum inhibitory concentration. As a result, the anti-thrombogenic coating may inhibit thrombus formation in portions of the vascular device otherwise prone to thrombus formation.

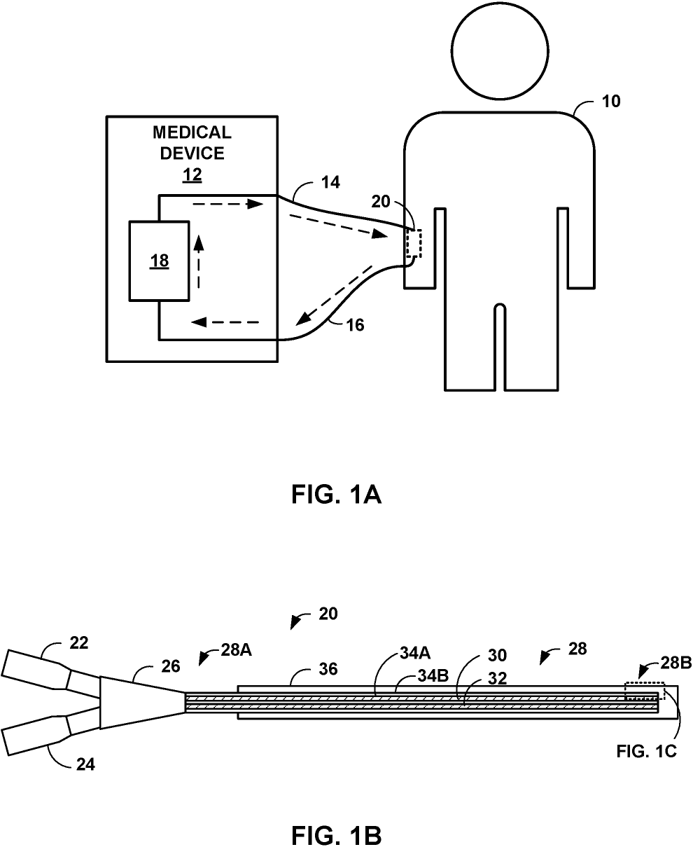

The anti-thrombogenic coatings may be applied to a variety of vascular devices used in a variety of systems. In some examples, anti-thrombogenic coatings described herein may be used with intravascular devices used in hemodialysis. For example, intravascular devices used for chronic vascular access may be kept within vasculature of a patient for extended periods of time, such that thrombi or other blood-based occlusions may be likely to form. FIG. 1A is a conceptual diagram of an example hemodialysis system and patient 10. However, the anti-thrombogenic coatings described herein may be used with other systems, e.g., in which an intravascular device remains in contact with blood for an extended period of time.

The dashed arrows indicate the direction of blood flow during a hemodialysis session. A clinician (or other user) may fluidically connect an aspiration line 16 to an inflow port (not shown) on a medical device 12 and to an intravascular device 20 in patient 10 to provide access to the vasculature of patient 10. Aspiration line 16 may be configured to facilitate the transport of blood from intravascular device 20 to medical device 12. For example, blood from patient 10 may contain high levels of waste products due to kidney failure or kidney disease. The clinician may also fluidically connect a perfusion line 14 to an outflow port (not shown) of the medical device 12 and to intravascular device 20 in patient 10 to provide access to a vein of patient 10. Perfusion line 14 may be configured to return relatively cleaner blood from medical device 12 to intravascular device 20.

Medical device 12 is configured to remove waste products from the blood received via aspiration line 16. For example, medical device 12 may include a dialyzer 26 and/or one or more filters that may remove waste products and excess fluid from the blood received via aspiration line 16. Dialyzer 26 may use a dialysate solution to remove the waste products and excess fluid from the blood of patient 10. Medical device 12 may also include a blood pump (not shown) which is configured to keep the blood of patient 10 flowing through medical device 12.

Intravascular device 20 may be a vascular access device, such as an intravenous catheter (e.g., to a lumen of the catheter), an arteriovenous fistula, or a synthetic graft (not shown). The arteriovenous fistula or the synthetic graft in the patient may be accessed, for example, via a needle or cannula. FIG. 1B is a cross-sectional conceptual diagram of an example hemodialysis catheter assembly as vascular device 20 of FIG. 1A that includes an anti-thrombogenic coating. Catheter assembly 20 includes an elongated body 28 and a proximal hub 26 that includes two ports - an aspiration port 22 and a perfusion port 24. Aspiration port 22 is configured to fluidically couple to aspiration line 16, while perfusion port 24 is configured to fluidically couple to perfusion line 14.

Elongated body 28 defines a proximal portion 28A, a distal portion 28B, and one or more lumens (e.g., two lumens for dialysis, three lumens for “trialysis”); in the example of FIG. 1B, elongated body 28 includes an aspiration lumen 30 fluidically coupled to the aspiration port 22 and a perfusion lumen 32 fluidically coupled to perfusion port 24. Elongated body 28 may be made of a synthetic resin, such as polyurethane, or other biocompatible material. In some embodiments, elongated body 28 is formed from a soft elastic material, such as silicone, which may require thicker walls because of the pliability of the silicone. Silicone may be preferred for certain applications because it is inert.

Elongated body 28 includes an inner surface 34A and an outer surface 34B (individually or collectively “surface 34” or “surfaces 34”). Inner surface 34A may include inner surfaces of aspiration lumen 30 and perfusion lumen 32. Inner surface 34A may be exposed to blood circulating through aspiration line 16 and perfusion line 14, while outer surface 34B may be exposed to blood within the vasculature of patient 10. Catheter assembly 20 may be placed in patient 10 for an extended period of time, such as on the order of weeks or months, such that, if left untreated, thrombi may form on at least a portion of inner surface 34A or outer surface 34B.

FIG. 1C is an expanded view of a distal portion of the catheter assembly 20 of FIG. 1B that includes an anti-thrombogenic coating 38. Catheter assembly 20 includes anti-thrombogenic coating 38 on at least a portion of at least one surface 34 of catheter assembly 20 to inhibit thrombus formation on catheter assembly 20. In some examples, anti-thrombogenic coating 38 may be present on portions of catheter assembly 20 that may contact blood for an extended period of time. For example, a distal portion of elongated body 28, which may be implanted in vasculature of patient 10, may include anti-thrombogenic coating 38. In some examples, anti-thrombogenic coating 38 may be directly adhered to surfaces 34, while in other examples, anti-thrombogenic coating 38 may be indirectly adhered to surfaces 34, such as through a bond coat or other intermediate layer or layers.

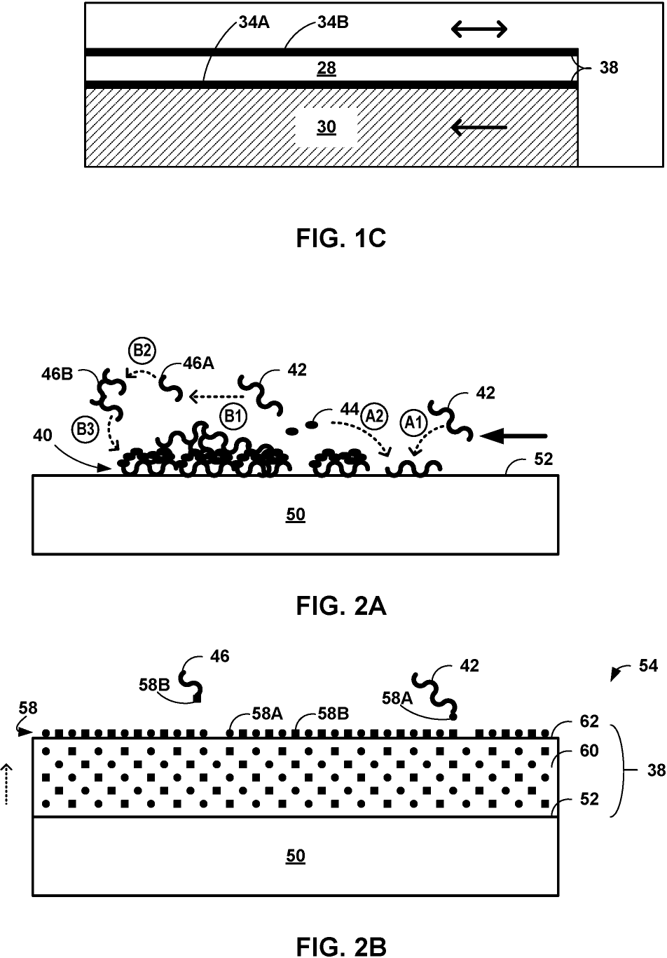

Anti-thrombogenic coating 38 includes one or more peptides configured to interact with fibrinogen or fibrinogen-derived proteins, such as fibrin, in blood and a polymer matrix configured to maintain a concentration of the one or more peptides at the surface above a minimum inhibitory concentration. Without being limited to any particular theory, FIG. 2A is a conceptual diagram illustrating fibrinogen-derived thrombus formation on a surface 52 of a substrate 50. In the example of FIG. 2A, substrate 50 may represent a portion of a vascular device, such as vascular device 20 of FIG. 1A, without an anti-thrombogenic coating. In some instances, fibrinogen 42 may be present in blood at a relatively high concentration, and may bind to surfaces of objects in the blood stream, such as surface 52 of substrate 50 (A1). When a vein becomes injured, the vein may release thrombin enzymes. Thrombin enzymes activate blood platelets 44, which bind to fibrinogen 42 and may aggregate on surface 52 (A2). Thrombin enzymes also convert fibrinogen 42 to soluble fibrin monomers 46A or other fibrinogen-derived proteins or fragments (B1). Fibrin monomers 46A polymerize to form insoluble fibrin polymers 46B (B2), which may migrate to surface 52 and form a mesh structure with the aggregated platelets 44 (B3), resulting in a thrombus 40.

In accordance with some examples of the disclosure, portions of medical devices that may be subject to thrombus formation may be coated with anti-thrombogenic coating 38 that includes one or more peptides configured to interact with fibrinogen 42 or fibrinogen-derived proteins, such as fibrin monomers 46A, in the blood to inhibit thrombus formation. FIG. 2B is a conceptual diagram illustrating inhibition of fibrinogen-derived thrombus formation using an anti-thrombogenic coating 38 on surface 52 of substrate 50. A portion of a medical device 54 includes an underlying substrate 50 and anti-thrombogenic coating 38 on surface 52 of substrate 50. Anti-thrombogenic coating 38 includes a polymer matrix 60 and one or more peptides 58A and 58B (individually and collectively “peptide 58” and “peptides 58”) dispersed in polymer matrix 60.

Peptides 58 are configured to interact with fibrinogen or fibrinogen-derived proteins, such as fibrin monomers 46A, in the blood at an outer surface 62 of anti-thrombogenic coating 38. Peptides 58 include at least one of a first type of peptides 58A or a second type of peptides 58B. In the example of FIG. 2B, anti-thrombogenic coating 38 includes both the first type of peptides 58A and the second type of peptides 58B. However, as will be described below, each of the first type of peptides 58A and the second type of peptides 58B may be capable of inhibiting thrombus formation, such that anti-thrombogenic coating 38 may only include one type of peptides 38, or may include a peptide that is capable of functioning as either a first type of peptide 58A or a second type of peptide 58B.

The first type of peptides 58A includes peptides configured to bind to fibrinogen 42. As described in FIG. 2A, fibrinogen 42 may bind to surface 52 of substrate 50 and subsequently bind to platelets 44 in the blood. The first type of peptides 58A may bind to fibrinogen 42 to reduce or prevent fibrinogen 42 from binding to surface 52 of substrate 50 (and/or surface 62 of coating 38), such as binding to platelets 44 deposited on surface 62. While not being limited to any particular theory, fibrinogen 42 may bind with particular receptors of platelets 44. In some examples, the first type of peptides 58A may include protein sequences that interfere with ligand-receptor interactions between peptides 58A and platelets 44 on surface 62, such as a protein sequence that is a close analog to a platelet binding site on fibrinogen 42. In some examples, the first type of peptides 58A includes at least one of glycyl-L-prolyl-L-arginyl-L-proline (GPRP), glycyl-L-prolyl-L-arginyl-L-valine-L-valine, or L-arginyl-glycyl-L-alpha-aspartyl-L-serine (RGDS). For example, the gly-pro-arg-pro sequence may bind to a terminal platelet binding site of an alpha-chain of a terminal domain of fibrinogen 42.

The second type of peptides 58B includes peptides configured to inhibit polymerization of fibrin monomers. As described in FIG. 2A, fibrinogen 42 may convert to fibrin 46 monomers and polymerize to form insoluble fibrin 46 polymer. The second type of peptides 58B may inhibit polymerization of fibrin to maintain fibrin 46 in solution and reduce or prevent fibrin 46 from forming a mesh structure with blood platelets 44. While not being limited to any particular theory, fibrinogen 42 may have two terminal domains. Thrombin may form fibrinogen-derived monomers, such as fibrin, by cleaving fibrinopeptides from one or more terminal ends of the terminal domains of fibrinogen 42 to expose a terminal polymerization site available for binding to other fibrin monomers. In some examples, the second type of peptides 58B may include protein sequences that are close analogs to one or more terminal polymerization sites of the fibrin monomers to bind to the terminal polymerization sites and inhibit polymerization. In some examples, the second type of peptides 58B includes at least one of glycyl-L-prolyl-L-arginyl-L-proline or glycyl-L-prolyl-L-arginyl-sarcosine. For example, the gly-pro-arg sequence may bind to a terminal polymerization site of an alpha-chain of a terminal domain of a fibrin monomer.

Peptides 58 may be present at surface 62 of anti-thrombogenic coating 38 and available to bind to fibrinogen 42. For example, peptides 58 may be exposed to blood at surface 62 and configured to release from surface 62 once bound to fibrinogen. As peptides 58 at surface 62 are used up, other peptides 58 may be exposed to blood at surface 62, such as through diffusion to surface 62 or degradation of polymer matrix 60 to expose other peptides 58 to blood at or near surface 62. For example, peptides 58 may bind to fibrinogen 42 while on surface 62 or in the bloodstream near surface 62, such that fibrinogen 62 may be inhibited from binding to surface 62.

Peptides 58 may be present at surface 62 of anti-thrombogenic coating 38 in a concentration that is at or above a minimum inhibitory concentration, such that a thrombus is unlikely to form on substrate 50 during contact with blood in vasculature of a patient. The minimum inhibitory concentration may represent a concentration at which peptides 58 may inhibit thrombus formation. The minimum inhibitory concentration of peptides 58 may be dependent on a variety of factors related to thrombus formation including, but not limited to, a concentration of fibrinogen in the blood, a concentration of thrombin in the blood, a flow rate of blood at substrate 50, an adhesion of surface 62 (e.g., a hydrophobicity of polymer matrix 60), a concentration of platelets in the blood, and the like. The minimum inhibitory concentration may be based on a concentration of fibrinogen in the blood near surface 62. For example, fibrinogen 42 may be present in a concentration between about 0.1 and 10 g/L, such as typically between about 1.5 and 4 g/L, and may interact with surface 62 based on concentration of fibrinogen 42 and, optionally, other factors such as molecular weight of fibrinogen 42, surface affinity of surface 62 (e.g., electrical charge), and various flow factors of the blood near surface 62. As such, the inhibitory concentration of peptides 58 may correspond to a surface density of peptides 58 at surface 62 that may inhibit fibrinogen for the particular concentration of fibrinogen 42 in the blood. In some examples, peptides 58 may be configured to maintain a presence at surface 62 above a 1:1 molar ratio of peptides 58 to fibrinogen 42 (e.g., MW~340,000 Da).

In some examples, the minimum inhibitory concentration may refer to a minimum inhibitory concentration for a particular mechanism of thrombus formation. For example, the first type of peptides 58A may be present at surface 62 at or above a minimum inhibitory concentration to inhibit binding of fibrinogen 42 to surface 62; additionally or alternatively, the second type of peptides 58B may be present at surface 62 at or above a minimum inhibitory concentration to inhibit polymerization of fibrin monomers 46A to fibrin polymers 46B. In some examples, the minimum inhibitory concentration may refer to a net minimum inhibitory concentration for a combination of mechanisms of thrombus formation. For example, the first type of peptides 58A and the second type of peptides 58B may be present at surface 62 at or above a net minimum inhibitory concentration to inhibit thrombus formation, such as through a combination of inhibiting binding of fibrinogen 42 to surface 62 and inhibiting polymerization of fibrin monomers 46B. Such a combination of the first type of peptides 58A and the second type of peptides 58B may enable inhibition of thrombus formation in a wider variety of individuals, for whom conditions for thrombus formation may differ.

In the example of FIG. 2B, polymer matrix 60 of coating 38 is configured to control release and/or exposure of peptides 58 to inhibit thrombus formation. Polymer matrix 60 may be configured to control a release rate of peptides 58 and a release time of peptides 58. The release rate of peptides 58 may represent a rate at which peptides 58 are replaced at surface 62. For example, the release rate of peptides may be related to a rate of diffusion of peptides to surface 62, a rate of degradation of polymer matrix 60, or a concentration of peptides 58 in polymer matrix 60.

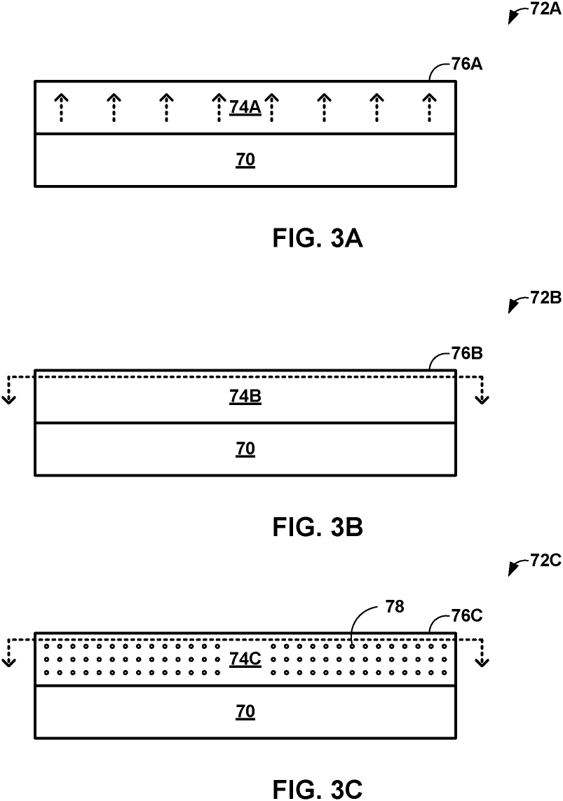

In some examples, polymer matrix 60 may be configured to provide (e.g., release or expose) peptides 58 at a release rate that is sufficient to maintain a concentration of peptides 58 at or near surface 62 at or above a minimum inhibitory concentration of peptides 58. A variety of parameters of polymer matrix 60 may be selected to achieve a desired release rate including, but not limited to, a minimum inhibitory concentration of peptides 58, a flow rate of blood at substrate 50, a concentration of peptides 58 in polymer matrix 60, and the like. FIGS. 3A-3C below illustrate various configurations of a coating 72A, 72B, 72C on a substrate 70, such as coating 52 on substrate 50.

In some examples, the polymer matrix is a diffusion-controlled polymer matrix. FIG. 3A is a conceptual diagram illustrating an anti-thrombogenic coating 72A that includes a diffusion-controlled polymer matrix 74A. Diffusion-controlled polymer matrix 74A may include any polymer matrix in which migration of dispersed peptides to a surface 76A may be controlled by diffusion of the peptides in polymer matrix 74A. For example, the peptides may diffuse from within polymer matrix 74A to surface 76A and/or into the blood. In such examples, a release rate of the peptides may at least be controlled by a concentration of the peptides in polymer matrix 74A and a composition of polymer matrix 74A. For example, as a concentration of peptides in polymer matrix 74A is increased or a molecular weight of polymers of polymer matrix 74A is decreased, a diffusion rate of the peptides from a bulk of polymer matrix 74A to surface 76A may increase. In some examples, the concentration of the peptides in polymer matrix 74A is at or greater than a saturation concentration of the peptides in polymer matrix 74A.

In some examples, diffusion-controlled polymer matrix 74A is a solvent-activated polymer matrix. A solvent-activated polymer matrix may include any polymer matrix in which migration of dispersed peptides to surface 76A may be controlled by swelling of polymer matrix 74A with a solvent and subsequent mobilization of the dispersed peptides. For example, a solvent, such as water, may migrate into polymer matrix 74A and swell polymer matrix 74A. This swelling of polymer matrix 74A may dissolve the peptides, such that the peptides may diffuse out of polymer matrix 74A to surface 76A and/or into the blood. In such examples, a release rate of the peptides may at least be controlled by a concentration of peptides in polymer matrix 74A, a bulk loading of peptides in polymer matrix 74A, a composition of the polymer of polymer matrix 74A, and the like. In some examples, the solvent is water absorbed from the blood, such that the solvent-activated polymer matrix is a hydrocolloid matrix. A variety of polymer may be used for the solvent-activated polymer matrix including, but not limited to, hydroxypropyl methylcellulose, dextrin, alginate, and the like.

In some examples, the polymer matrix is a bioabsorbable polymer matrix. FIG. 3B is a conceptual diagram illustrating an anti-thrombogenic coating 72B that includes a biodegradable polymer matrix 74B. Bioabsorbable polymer matrix 74B may include any polymer matrix in which access to dispersed peptides at a surface 76B may be controlled by degradation of polymer matrix 74B and subsequent mobilization of the dispersed peptides. For example, water may migrate into polymer matrix 74B and react with polymer matrix 74B to break covalent bonds, thereby exposing the peptides to surface 76B and regenerating a concentration of peptides at surface 76B. A variety of bioabsorbable polymers may be used for the bioabsorbable polymer matrix including, but not limited to, poly(lactic acid) (PLA), poly(glycolic acid) (PGA), and the like.

In some examples, polymer matrix 74B is a conjugated polymer matrix. A conjugated polymer matrix may include any polymer matrix in which exposure or release of peptides from surface 76B may be controlled by breaking chemical bonds between polymer matrix 74B and the peptides. For example, polymer matrix 74B may be functionalized with peptides. When the peptides at surface 76B bonds with fibrinogen, the peptide may release from surface 76B. In such examples, a release rate of peptides may at least be controlled by a composition of polymer matrix 74B, a strength of bonding between polymer matrix 74B and peptides, a composition of a reactive moiety bonding peptides to polymer matrix 74B, and the like. A variety of polymers may be used for the conjugated polymer matrix including, but not limited to, polyethylene glycol (PEG), and the like.

In some examples, the peptides may be encased in a bioabsorbable polymer shell that form microspheres that are incorporated into a polymer matrix. FIG. 3C is a conceptual diagram illustrating an anti-thrombogenic coating 72C that includes microspheres 78 in a biodegradable polymer matrix 74C. For example, microspheres 78 are dispersed in a bioabsorbable polymer matrix 74C and configured to become exposed to the blood through degradation of polymer matrix 74C. Further, the bioabsorbable polymer shell of microspheres 78 may degrade to release peptides. As such, properties of both polymer matrix 74C and the bioabsorbable polymer shell of microspheres 78 may be selected to tune a release rate of peptides to surface 76C. In such examples, a release rate of peptides may at least be controlled by a composition of polymer matrix 74C, a composition of the bioabsorbable polymer shell of microspheres 78, a thickness of polymer matrix 74C, a concentration of microspheres 78 in polymer matrix 74C, a size of microspheres 78, a thickness of the bioabsorbable polymer shell of microspheres 78, and the like.

Referring back to FIG. 2B, in some examples, polymer matrix 60 may be configured to release peptides 58 for a release time that is sufficient to maintain the release rate for a particular amount of time. For example, substrate 50 may be placed in vasculature of a patient for a set amount of time, such that polymer matrix 60 may be configured with a loading of peptides 58 that will release peptides for the set amount of time. A variety of parameters of polymer matrix 60 may be selected to achieve a desired release time including, but not limited to, a release rate of peptides 58 from polymer matrix 60, a concentration of peptides 58 in polymer matrix 60, a thickness of polymer matrix 60, a surface area of anti-thrombogenic coating 38, and the like. In some examples, polymer matrix 60 may have a thickness between about 5 µm and about 100 µm.

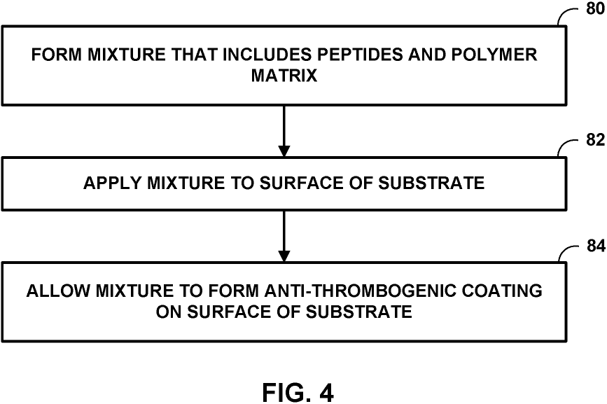

FIG. 4 is a flowchart illustrating an example technique for forming an anti-thrombogenic coating on a vascular device. However, other techniques may be used to form the anti-thrombogenic coating on the vascular device, such as polymer grafting techniques. The example technique of FIG. 4 will be described with reference to FIG. 2B.

The method of FIG. 4 includes forming a mixture that includes one or more peptides 58 and polymer matrix (80). Polymer matrix 60 may be selected and configured to control access to and/or release of peptides 58. As described above, peptides 58 are configured to interact with fibrinogen or fibrinogen-derived proteins in the blood. In some examples, the mixture may include at least one of first type of peptides 58A configured to bind to the fibrinogen 42 or second type of peptides 58B configured to inhibit polymerization of fibrin monomers 46A. In some examples, the mixture may include a hydrocolloid polymer matrix 60 that includes peptides 58 and is configured to gel and encapsulate peptides 58 prior to or after application of the mixture to a surface. In some examples, polymer matrix 60 may be a bioabsorbable polymer matrix configured to degrade at a desired degradation rate. The degradation rate of the polymer matrix may correspond to a rate at which peptides 58 may be replenished at surface 62 of coating 38. In such examples, peptides 58 may be conjugated to polymer matrix 60 after polymerization of monomers to form polymer matrix 60.

In some examples, peptides 58 may be encapsulated in a bioabsorbable polymer shell prior to being incorporated into the mixture. For example, peptides 58 may be emulsified in a surfactant and first monomer mixture. The monomer of the first monomer mixture may be configured to form a polymer shell that degrades a particular rate, such as described above. The first monomer mixture may be polymerized to form microspheres, which may be subsequently dispersed in a second monomer mixture. In such examples, the monomer in the second monomer mixture may be selected to form a bioabsorbable polymer matrix configured to expose the microspheres at a desired release rate. The release rate may correspond to a rate at which microspheres of peptides 58 may be replenished at surface 62 of coating 38. In this way, both a degradation rate of the polymer shell of the microspheres and a release rate of the bulk polymer matrix 60 may be controlled to tune coating 38 to provide peptides 58 above the minimum inhibitory concentration.

The method of FIG. 4 includes applying a mixture to a surface of a vascular device (82). For example, the mixture of polymer matrix 60 and peptides 58 may be applied to the surface of the medical device. The surface of the medical device may include any surface of the medical device for which a thrombus may be likely to form. For example, thrombus formation may be dependent on various conditions within a blood vessel, such as flow rate of blood. As a result, some portions of the vasculature may be more prone to thrombus formation on corresponding portions of vascular device. In examples in which the vascular device is a catheter, the mixture may be applied to a distal portion of the catheter, and may include application to an outer surface of the elongated body of the catheter or an inner surface of one or more lumens of the catheter.

The method of FIG. 4 includes allowing the mixture to form anti-thrombogenic coating 38 (84). For example, the mixture may cure, harden, gel, or undergo another posttreatment process to form anti-thrombogenic coating 38. Polymer matrix 60 is configured to control release of the one or more peptides 58, such as by providing peptides 58 at a surface of polymer matrix 60 as described above.

Example 1: A medical device includes a vascular device; and an anti-thrombogenic coating on a surface of the vascular device, wherein the anti-thrombogenic coating comprises: one or more peptides configured to interact with fibrinogen and/or fibrinogen-derived proteins in blood; and a polymer matrix configured to control availability of the one or more peptides at a surface of the anti-thrombogenic coating.

Example 2: The medical device of example 1, wherein the polymer matrix is configured to maintain a concentration of the one or more peptides at the surface of the anti-thrombogenic coating above a minimum inhibitory concentration.

Example 3: The medical device of example 1 or 2, wherein the one or more peptides comprise at least one of: a first type of peptides configured to bind to the fibrinogen; or a second type of peptides configured to inhibit polymerization of the fibrinogen-derived proteins.

Example 4: The medical device of example 3, wherein the polymer matrix is configured to maintain at least one of: a concentration of the first type of peptides at the surface of the anti-thrombogenic coating above a first minimum inhibitory concentration corresponding to inhibition of binding of fibrinogen to the surface of the anti-thrombogenic coating; or a concentration of the second type of peptides at the surface of the anti-thrombogenic coating above a second minimum inhibitory concentration corresponding to inhibition of polymerization of the fibrinogen-derived proteins.

Example 5: The medical device of example 3 or 4, wherein the one or more peptides comprise both the first type of peptides and the second type of peptides.

Example 6: The medical device of any of examples 3 to 5, wherein the first type of peptides comprises at least one of glycyl-L-prolyl-L-arginyl-L-proline (GPRP), glycyl-L-prolyl-L-arginyl-L-valine-L-valine, or L-arginyl-glycyl-L-alpha-aspartyl-L-serine (RGDS).

Example 7: The medical device of any of examples 3 to 6, wherein the second type of peptides comprises at least one of glycyl-L-prolyl-L-arginyl-L-proline or glycyl-L-prolyl-L-arginyl-sarcosine.

Example 8: The medical device of any of examples 1 to 7, wherein the polymer matrix comprises a bioabsorbable polymer matrix.

Example 9: The medical device of example 8, wherein the one or more peptides are conjugated to the bioabsorbable polymer matrix.

Example 10: The medical device of any of examples 1 to 9, wherein the one or more peptides are encapsulated in polymer shells as microspheres, and wherein the microspheres are dispersed in the polymer matrix.

Example 11: The medical device of example 10, wherein the polymer matrix comprises a hydrocolloid polymer matrix.

Example 12: The medical device of any of examples 1 to 11, wherein the vascular device comprises a catheter comprising an elongated body and defining at least one lumen, and wherein the surface of the vascular device comprises a distal portion of the catheter.

Example 13: A hemodialysis medical assembly includes a hemodialysis catheter assembly configured to fluidically couple to a hemodialysis machine, wherein the hemodialysis catheter assembly comprises: a catheter includes one or more peptides configured to interact with fibrinogen or fibrinogen-derived proteins in blood; and a polymer matrix configured to control availability of the one or more peptides at a surface of the anti-thrombogenic coating.

Example 14: The hemodialysis medical assembly of example 13, wherein the one or more peptides comprise at least one of: a first type of peptides configured to bind to the fibrinogen; or a second type of peptides configured to inhibit polymerization of the fibrinogen-derived proteins.

Example 15: The hemodialysis medical assembly of example 13 or 14, wherein the surface of the catheter comprises an outer surface of the elongated body, an inner surface of the aspiration lumen, and an inner surface of the perfusion lumen.

Example 16: The hemodialysis medical assembly of any of examples 13 to 15, further comprising the hemodialysis machine configured to filter waste products from the blood.

Example 17: A method includes forming an anti-thrombogenic coating on a surface of a vascular device, wherein the anti-thrombogenic coating comprises: one or more peptides configured to interact with fibrinogen and/or fibrinogen-derived proteins in blood; and a polymer matrix configured to control availability of the one or more peptides at a surface of the anti-thrombogenic coating.

Example 18: The method of example 17, further comprising: applying a mixture to the surface of the vascular device, wherein the mixture comprises the one or more peptides and the polymer matrix; and curing the mixture to form the anti-thrombogenic coating.

Example 19: The method of example 18, further comprising, prior to applying the mixture to the surface, encapsulating the one or more peptides into a bioabsorbable polymer shell as microspheres.

Example 20: The method of any of examples 17 to 19, wherein the one or more peptides comprise at least one of: a first type of peptides configured to bind to the fibrinogen; or a second type of peptides configured to inhibit polymerization of the fibrinogen-derived proteins.

Example 21: The method of any of examples 17 to 20, wherein the vascular device comprises a catheter comprising an elongated body and defining at least one lumen, and wherein the surface comprises a distal portion of the catheter.

Various examples have been described. These and other examples are within the scope of the following claims.

What is claimed is:

1. A medical device comprising:
a vascular device; and
an anti-thrombogenic coating on a surface of the vascular device, wherein the anti-thrombogenic coating comprises:
one or more peptides configured to interact with fibrinogen and/or fibrinogen-derived proteins in blood; and
a polymer matrix configured to control availability of the one or more peptides at a surface of the anti-thrombogenic coating.

2. The medical device of claim 1, wherein the polymer matrix is configured to maintain a concentration of the one or more peptides at the surface of the anti-thrombogenic coating above a minimum inhibitory concentration.

3. The medical device of claim 1, wherein the one or more peptides comprise at least one of:
a first type of peptides configured to bind to the fibrinogen; or
a second type of peptides configured to inhibit polymerization of the fibrinogen-derived proteins.

4. The medical device of claim 3, wherein the polymer matrix is configured to maintain at least one of:
a concentration of the first type of peptides at the surface of the anti-thrombogenic coating above a first minimum inhibitory concentration corresponding to inhibition of binding of fibrinogen to the surface of the anti-thrombogenic coating; or
a concentration of the second type of peptides at the surface of the anti-thrombogenic coating above a second minimum inhibitory concentration corresponding to inhibition of polymerization of the fibrinogen-derived proteins.

5. The medical device of claim 3, wherein the one or more peptides comprise both the first type of peptides and the second type of peptides.

6. The medical device of claim 3, wherein the first type of peptides comprises at least one of glycyl-L-prolyl-L-arginyl-L-proline (GPRP), glycyl-L-prolyl-L-arginyl-L-valine-L-valine, or L-arginyl-glycyl-L-alpha-aspartyl-L-serine (RGDS).

7. The medical device of claim 3, wherein the second type of peptides comprises at least one of glycyl-L-prolyl-L-arginyl-L-proline or glycyl-L-prolyl-L-arginyl-sarcosine.

8. The medical device of claim 1, wherein the polymer matrix comprises a bioabsorbable polymer matrix.

9. The medical device of claim 8, wherein the one or more peptides are conjugated to the bioabsorbable polymer matrix.

10. The medical device of claim 1, wherein the one or more peptides are encapsulated in polymer shells as microspheres, and wherein the microspheres are dispersed in the polymer matrix.

11. The medical device of claim 10, wherein the polymer matrix comprises a hydrocolloid polymer matrix.

12. The medical device of claim 1, wherein the vascular device comprises a catheter comprising an elongated body and defining at least one lumen, and wherein the surface of the vascular device comprises a distal portion of the catheter.

13. A hemodialysis medical assembly comprising:
a hemodialysis catheter assembly configured to fluidically couple to a hemodialysis machine, wherein the hemodialysis catheter assembly comprises:
a catheter comprising an elongated body and defining an aspiration lumen and a perfusion lumen; and
an anti-thrombogenic coating on a surface of the catheter, wherein the anti-thrombogenic coating comprises:
one or more peptides configured to interact with fibrinogen or fibrinogen-derived proteins in blood; and
a polymer matrix configured to control availability of the one or more peptides at a surface of the anti-thrombogenic coating.

14. The hemodialysis medical assembly of claim 13, wherein the one or more peptides comprise at least one of:
a first type of peptides configured to bind to the fibrinogen; or
a second type of peptides configured to inhibit polymerization of the fibrinogen-derived proteins.

15. The hemodialysis medical assembly of claim 13, wherein the surface of the catheter comprises an outer surface of the elongated body, an inner surface of the aspiration lumen, and an inner surface of the perfusion lumen.

16. A method comprising:
forming an anti-thrombogenic coating on a surface of a vascular device, wherein the anti-thrombogenic coating comprises:
one or more peptides configured to interact with fibrinogen and/or fibrinogen-derived proteins in blood; and
a polymer matrix configured to control availability of the one or more peptides at a surface of the anti-thrombogenic coating.

17. The method of claim 16, further comprising:
applying a mixture to the surface of the vascular device, wherein the mixture comprises the one or more peptides and the polymer matrix; and
curing the mixture to form the anti-thrombogenic coating.

18. The method of claim 17, further comprising, prior to applying the mixture to the surface, encapsulating the one or more peptides into a bioabsorbable polymer shell as microspheres.

19. The method of claim 16, wherein the one or more peptides comprise at least one of:
a first type of peptides configured to bind to the fibrinogen; or
a second type of peptides configured to inhibit polymerization of the fibrinogen-derived proteins.

20. The method of claim 16, wherein the vascular device comprises a catheter comprising an elongated body and defining at least one lumen, and wherein the surface comprises a distal portion of the catheter.
